(19) United States
(12) Patent Application Publication
(10) Pub. No.: US 2025/0213851 A1
(43) Pub. Date: Jul. 3, 2025

(54) ASYMMETRIC OVAL-SHAPED AND ROUNDED TRIANGULAR-SHAPED ELECTRODE ASSEMBLIES

(71) Applicant: Novocure GmbH, Root D4 (CH)

(72) Inventors: Elie YAACOBI, Haifa (IL); Noa HALAVEE, Tel Aviv (IL); Dmitry GOLOM, Haifa (IL)

(73) Assignee: Novocure GmbH, Root D4 (CH)

(21) Appl. No.: 19/002,817

(22) Filed: Dec. 27, 2024

Related U.S. Application Data
(60) Provisional application No. 63/615,891, filed on Dec. 29, 2023.
(60) Provisional application No. 63/567,719, filed on Mar. 20, 2024.

(51) Int. Cl. A61N 1/04; A61N 1/36

(52) U.S. Cl. CPC A61N 1/0496; A61N 1/0408; A61N 1/36002

(57) ABSTRACT

An electrode assembly for delivering tumor treating fields to a subject's body, the electrode assembly apparatus including a layer of conductive anisotropic material, an electrode element and a cover. The electrode assembly is provided in different shapes, such as an asymmetric oval, including ovaloid, ovoid and ovate shapes or stretched asymmetric oval, ovaloid, ovoid, or ovate shapes, as well as a pear shape and a rounded arrowhead shape. These shapes can be provided in different sizes to allow the electrode assembly to be attached or secured to a subject or patient's body, especially when the site may be nonplanar, or otherwise contoured, resulting in a more comfortable experience, or even a more favorable clinical outcome.

### CROSS-REFERENCE TO RELATED APPLICATIONS

This application claims priority to U.S. Provisional Application No. 63/615,891, filed Dec. 29, 2023, and U.S. Provisional Application No. 63/567,719, filed Mar. 20, 2024, the contents of each are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the subject's body and applying alternating current (AC) voltages between the transducers. Conventionally, transducers used to generate TTFields include a plurality of electrode elements comprising ceramic disks. One side of each ceramic disk is positioned against the subject's skin, and the other side of each disc has a conductive backing. Electrical signals are applied to this conductive backing, and these signals are capacitively coupled into the subject's body through the ceramic discs. Conventional transducer designs include rectangular arrays of ceramic disks aligned with each other in straight rows and columns and attached to the subject's body via adhesive.

### SUMMARY

Disclosed herein, in various aspects, are electrode assemblies and methods for delivering electric fields, for example alternating electric fields, such as TTFields, to a subject.

In one aspect, an electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

In another aspect, an electrode assembly for delivering an alternating electric field to a subject, the electrode assembly comprising: an electrode layer having a front face and a back face, the front face of the electrode layer adapted to face toward the subject, the back face being opposite to the front face, wherein, when viewed in a direction perpendicular to the front face of the electrode layer, the electrode layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

In yet another aspect, an electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer comprises: a first edge, a second edge, and a third edge in a triangular configuration; a first rounded vertex between the first edge and second edge, a second rounded vertex between the second edge and third edge; and a third rounded vertex between the third edge and first edge.

### BRIEF DESCRIPTION OF THE DRAWINGS

FIG. 1 depicts an example of an electrode assembly having a stretched asymmetric oval shape.

FIG. 2 depicts an exploded view of the example of the electrode assembly of FIG. 1.

FIG. 3 depicts an example of an electrode assembly having an asymmetric oval or ovate shape.

FIG. 4 depicts an exploded view of the example of the electrode assembly of FIG. 3.

FIG. 5 depicts an example of an electrode assembly having a rounded triangular shape.

FIG. 6 depicts an exploded view of the example of the electrode assembly of FIG. 5.

FIG. 7 depicts an example of an electrode assembly having a rounded arrowhead shape.

FIG. 8 depicts an exploded view of the example of the electrode assembly of FIG. 7.

FIGS. 9A-9D depict different sizes of the electrode assembly having the stretched asymmetric oval shape of FIG. 1.

FIGS. 10A-10D depict different sizes of the electrode assembly having the asymmetric oval or ovate shape of FIG. 3.

FIGS. 11A-11D depict different sizes of the electrode assembly having the rounded triangular shape of FIG. 5.

FIGS. 12A-12D depict different sizes of the electrode assembly having the rounded arrowhead shape of FIG. 7.

FIGS. 13A-13G depict different examples of electrode layers.

FIGS. 14A-14I depict examples of electrode assemblies.

FIGS. 15A-15C depict positioning of electrode assemblies on a subject's body for application of TTFields.

FIG. 16 depicts an example of a conductive anisotropic layer.

FIG. 17 depicts an example of an electrode layer.

FIGS. 18A-18D depict different sizes of an electrode assembly.

FIGS. 19A-19D depict different sizes of an electrode assembly.

FIG. 20 depicts an example of an electrode assembly.

FIG. 21 depicts an example of an electrode assembly.

Various embodiments are described in detail below with reference to the accompanying drawings, wherein like reference numerals represent like elements.

### DESCRIPTION OF EMBODIMENTS

This application describes exemplary electrode assembly apparatuses used to apply TTFields to a subject's body for treating one or more cancers. This application also describes exemplary methods to apply TTFields to a subject's body using electrode assemblies.

Electrode assemblies used to apply TTFields to a subject's body may include multiple electrode elements electrically coupled together on a substrate and attached to the subject's body at a desired location, for example, via an adhesive backing of the substrate or top cover (e.g., an overlay bandage) or a separately applied adhesive. Electrode assemblies may have large, rectangular surfaces so as to maximize a number of electrode elements that are located on the electrode assembly for applying TTFields to the subject's body. However, as recognized by the inventors, the use of such electrode assemblies may be disadvantageous in the treatment of patients with variations in bodily contours, such as uneven and non-planar body portions.

The inventors have now recognized that a need exists for electrode assemblies that can accommodate stretching/twisting of the body, especially on the torso, as well as variations in contours of the body which may occur near the areas where TTFields are applied. The use of asymmetric electrode assemblies which can conform to the contours of the patient's body may result in both a more comfortable patient experience as well as a better clinical outcome. Shapes of the electrode assemblies that have been found to be advantageous with respect to comfort (and may also be reflected in better clinical outcomes) include asymmetric ovals, ovaloids, 2-dimensional ovoids, ovates, or stretched asymmetric ovals, rounded triangular shapes, rounded arrowhead, and pear shapes. As used herein, the term “stretched oval, ovaloid, ovoid, or ovate shape” (or any reordering thereof) may refer to “stretched oval, stretched ovaloid, stretched ovoid, or stretched ovate shape;” the term “asymmetric oval, ovaloid, ovoid, or ovate shape” (or any reordering thereof) may refer to “asymmetric oval, asymmetric ovaloid, asymmetric ovoid, or asymmetric ovate shape;” and the term “stretched asymmetric oval, ovaloid, ovoid, or ovate” (or any reordering thereof) may refer to “stretched asymmetric oval, stretched asymmetric ovaloid, stretched asymmetric ovoid, or stretched asymmetric ovate.”

Ovals can have just one axis of symmetry (mirror plane), which can be viewed as egg-shaped (2-dimensional); or ovals can have two axes of symmetry (two perpendicular mirror planes), which are usually referred to as an ellipse. Herein, the term “asymmetric oval” refers to the former: ovals having just one axis of symmetry (egg-shaped). Herein, the term “stretched asymmetric oval” describes a shape similar to the asymmetric oval (above), which also has one axis of symmetry (mirror plane), but also includes a straight-line portion on each of the longer edges. Accordingly, herein, the “asymmetric oval” is generally egg-shaped (in 2-dimensions) and has convex arcs around the perimeter and one axis of symmetry; whereas the “stretched asymmetric oval” is the convex hull of two circles of dissimilar radii connected by their external tangent lines (an asymmetric oval, as above, but with straight line portions on opposing longer edges between two dissimilar convex ends). The length of the straight line portions of the longer edges of the stretched asymmetric oval may be the same for the two edges of the stretched asymmetric oval, but the length can, of course, vary from one stretched asymmetric oval to another. That is, figuratively, the stretched asymmetric oval can be stretched to a different extent.

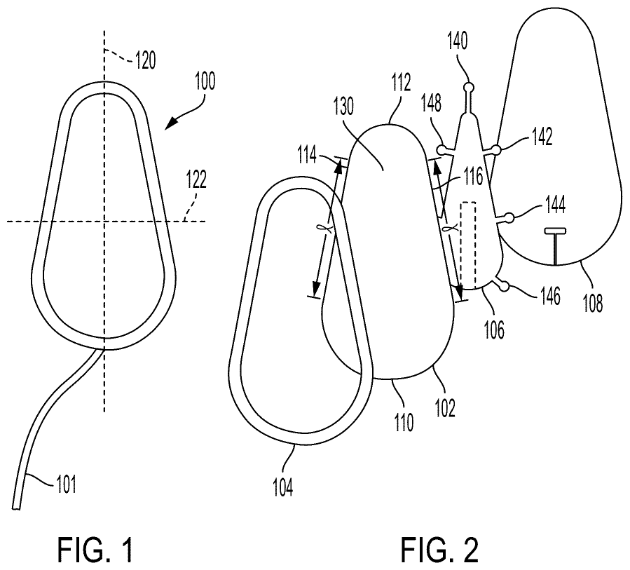

FIG. 1 depicts an example of an electrode assembly 100 having a stretched asymmetric oval shape, and FIG. 2 depicts an exploded view of the electrode assembly 100 of FIG. 1. The electrode assembly 100 may include a foam or tape outline 104, a conductive anisotropic material layer 102, an electrode layer 106, a top cover 108, and an electrical power connection 101. When placed on a subject to apply an electric field to a subject, the foam or tape outline 104 may be closest to the subject, and the top cover 108 may be furthest from the subject. When viewed in a direction perpendicular to a face of the electrode assembly 100 facing the subject, the electrode assembly 100 may have a stretched asymmetric oval or stretched ovaloid shape (or in some embodiments, a stretched ovoid shape or a stretched ovate shape).

The conductive anisotropic material layer 102 may be electrically coupled to the electrode layer 106. The conductive anisotropic material layer 102 may have a front face 130 and a back face, where the back face of the conductive anisotropic material layer 102 may face toward the electrode layer 106, and where the front face 130 of the conductive anisotropic material layer 102 may be adapted to face toward the subject. The conductive anisotropic material layer 102, discussed further below, may be a material such as, but not limited to, a sheet of graphite. In some embodiments, the conductive anisotropic material layer 102 may be sandwiched between a first conductive adhesive or hydrogel layer (towards the front face 130) and a second conductive adhesive or hydrogel layer (towards the back face).

When viewed in a direction perpendicular to the front face 130 of the conductive anisotropic material layer 102, the conductive anisotropic material layer 102 may have a stretched asymmetric oval or ovaloid shape (or in some embodiments, a stretched ovoid shape or a stretched ovate shape). The conductive anisotropic material layer 102 may have a first end 110 and a second end 112. The first end 110 may be referred to as a basal end, and the second end 112 may be referred to as an apical end. The first end 110 may have a first radius of curvature, and the second end 112 may be opposite the first end 110 and have a second radius of curvature. The first radius of curvature of the first end 110 may be greater than the second radius of curvature of the second end 112. The conductive anisotropic material layer 102 may have a mirror symmetry configuration about a major axis of symmetry 120 and an asymmetric configuration about a minor axis of symmetry 122 perpendicular to the major axis (for clarity, shown in FIG. 1). A typical length of the conductive anisotropic material layer 102 along the major axis of symmetry 120 may be about 10 cm to about 45 cm, or may be at least 10 cm and at most 45 cm, or may be about 10 cm to about 35 cm, or may be at least 10 cm and at most 35 cm.

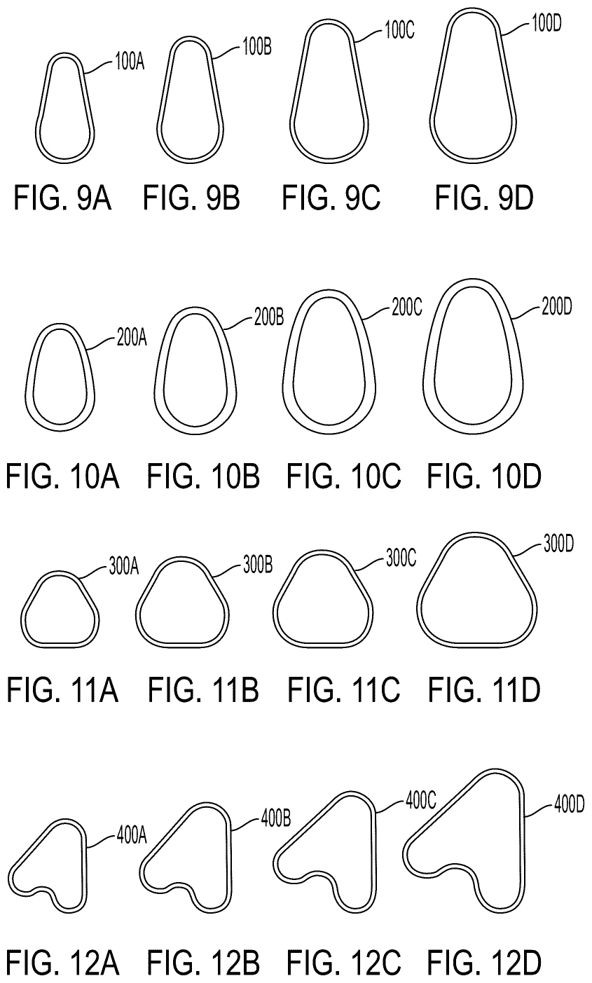

The conductive anisotropic material layer 102 may have edges 114, 116 extending from the first end 110 to the second end 112. In some embodiments, the edges 114, 116 may be relatively straight, such that the conductive anisotropic material layer 102 may have a stretched asymmetric oval or stretched ovaloid shape. Each of the edges 114, 116 may have a straight line portion of length l. The length l of the straight line portions of the edges 114, 116 may vary from one stretched asymmetric oval to another. As an example, as illustrated in FIGS. 9A-9D, the stretched asymmetric oval shapes have straight line portions on the longer edges of different lengths.

The electrode layer 106 may be positioned on the back face of the conductive anisotropic material layer 102. The electrode layer 106 may comprise a flexible printed circuit board (PCB). The electrode layer 106 may be a flexible printed circuit board (PCB) with pads or substantially planar pieces of metal attached (electrically connected) thereto. The electrode layer 106 may include a polymer film that is adapted to deliver an alternating electric field. The electrode layer 106 may include polymer films disposed over pads on a printed circuit board or over substantially planar pieces of metal. In some embodiments, the polymer film may have a high dielectric constant, such as, for example, a dielectric constant greater than 10, or, in some embodiments, greater than 20. The electrode layer 106 may be sandwiched between the conductive anisotropic material layer 102 and the top cover 108. In some embodiments, an area enclosed by the perimeter of the conductive anisotropic material layer 102 may be at least 10% larger, at least 20% larger, at least 30% larger, at least 40% larger, at least 50% larger, at least 60% larger, at least 70% larger, or at least 80% larger, than an area enclosed by the perimeter of the electrode layer 106. In some embodiments, a shape of the electrode layer 106 may be similar to, but smaller than, a shape of the conductive anisotropic material layer 102. For example, in some embodiments, the electrode layer 106 may have a stretched asymmetric oval, ovaloid, ovoid, or ovate shape. In some embodiments, the electrode layer 106 may have an asymmetric oval, ovaloid, ovoid, or ovate shape. In this way, the electrode layer 106 may include a first radius of curvature and a second radius of curvature, each of which is smaller than the first and second radii of curvature, respectively, of the conductive anisotropic material layer 102. The electrode layer 106 may include protrusions about the periphery thereof (for example, including protrusions 140, 142, 144, 146, and 148 as shown in FIG. 2). The protrusions 140, 142, 144, 146, and 148 may include thermistors positioned thereon. The number and distribution of the protrusions and/or thermistors may vary. The electrode layer 106 may have one, two, three, or any other number of electrode elements depending on the size and shape of the electrode layer 106.

The foam or tape outline 104 may be attached or secured to a periphery of the front face 130 of the conductive anisotropic material layer 102. The foam or tape outline 104 may include an adhesive layer on its front (skin-facing) side, which may aid in securing the electrode assembly in place on the subject.

The top cover 108 may provide support for the electrode assembly 100. The top cover 108 may be a flexible plastic or fabric or other flexible material. In some embodiments, it may be an adhesive-coated overlay tape or bandage extending laterally beyond the perimeter of the conductive anisotropic material layer 102 whereby the electrode assembly is adhered to the subject's body.

The electrical power connection 101 may be electrically connected to the electrode layer 106 via the top cover 108. The electrical power connection 101 may be attached to the electrode assembly 100 and, in some embodiments, may be removably attached to the electrode assembly 100.

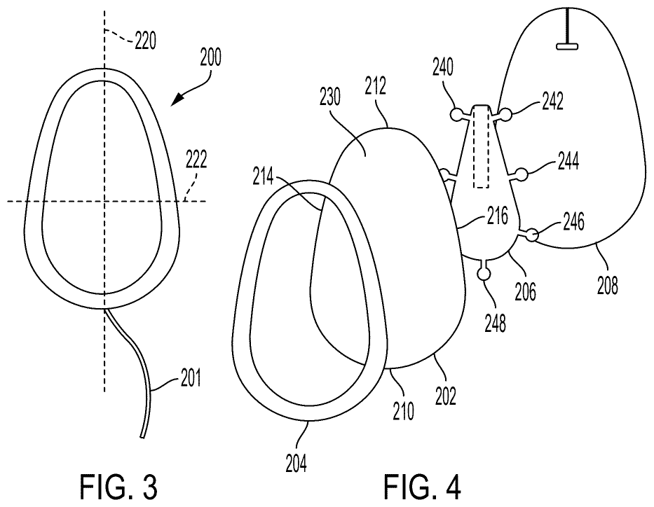

FIG. 3 depicts an example of an electrode assembly 200 having an ovate shape, and FIG. 4 depicts an exploded view of the example of the electrode assembly 200 of FIG. 3. The electrode assembly 200 is similar to the electrode assembly 100, but the electrode assembly 200 may have an ovate shape, instead of a stretched asymmetric oval or ovaloid shape of the electrode assembly 100. The electrode assembly 200 may include a foam or tape outline 204, a conductive anisotropic material layer 202, an electrode layer 206, a top cover 208, and an electrical power connection 201, which may be similar to the foam or tape outline 104, conductive anisotropic material layer 102, electrode layer 106, top cover 108, and electrical power connection 101, except for their shapes. When placed on a subject to apply an electric field to a subject, the foam or tape outline 204 may be closest to the subject, and the top cover 208 may be furthest from the subject. When viewed in a direction perpendicular to a face of the electrode assembly 200 facing the subject, the electrode assembly 200 may have an ovate shape.

The conductive anisotropic material layer 202 may be similar to the conductive anisotropic material layer 102, except for its shape. In some embodiments, similar to the similarly named and/or numbered features of the conductive anisotropic material layer 102, the conductive anisotropic material layer 202 may include a front face 230, a back face, a first end 210 having a first radius of curvature, a second end 212 having a second radius of curvature, edges 214, 216, a mirror symmetry configuration about a major axis of symmetry 220, and an asymmetric configuration about a minor axis of symmetry 222 perpendicular to the major axis (for clarity, shown in FIG. 3). However, when viewed in a direction perpendicular to the front face 230 of the conductive anisotropic material layer 202, the conductive anisotropic material layer 202 may have an asymmetric oval, ovaloid or ovate shape (or in some embodiments, an asymmetric ovoid shape). The edges 214, 216 of the conductive anisotropic material layer 202 may have a convex or bulging shape, such that the conductive anisotropic material layer 202 may have an asymmetric oval, ovaloid or ovate shape (or an ovoid shape). The edges 214, 216 of the conductive anisotropic material layer 202 may bulge away from the center of the conductive anisotropic material layer 202.

The foam or tape outline 204, the electrode layer 206, the top cover 208, and the electrical power connection 201 may be similar to the foam or tape outline 104, the electrode layer 106, the top cover 108, and the electrical power connection 101. However, the shape of the foam or tape outline 204, the electrode layer 206, and the top cover 208 may be similar to the shape of the conductive anisotropic material layer 202, namely an asymmetric oval, ovaloid, ovoid or ovate shape. For example, in some embodiments, the shape of the foam or tape outline 204, the electrode layer 206, and the top cover 208 may be an asymmetric oval, ovaloid, ovoid, or ovate shape. Similar to the electrode layer 106, the electrode layer 206 may include a number of protrusions about the periphery thereof (for example, including protrusions 240, 242, 244, 246, and 248, as shown in FIG. 4), which may include thermistors positioned thereon.

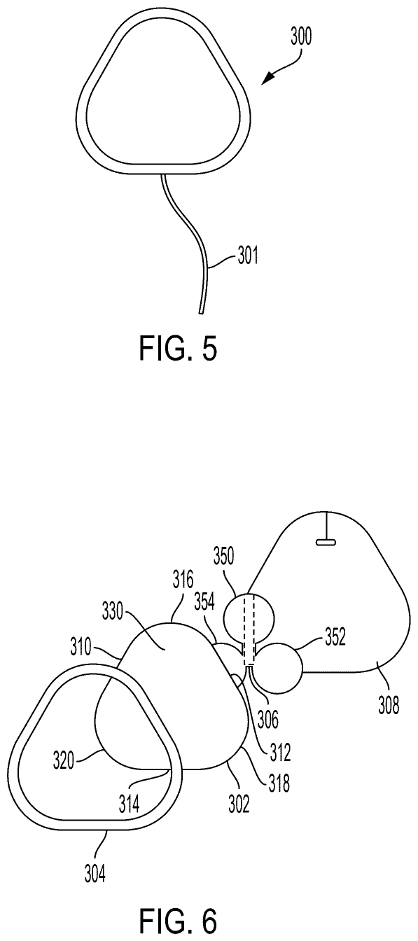

FIG. 5 depicts an example of an electrode assembly 300 having a rounded triangular shape, and FIG. 6 depicts an exploded view of the example of the electrode assembly 300 of FIG. 5. The electrode assembly 300 may be similar to the electrode assemblies 100 and 200, but the electrode assembly 300 may have a rounded triangular shape, instead of a stretched asymmetric oval, ovaloid or ovate shape or an asymmetric oval, ovaloid or ovate shape of the electrode assemblies 100 and 200. A rounded triangular shape has a generally triangular shape but has rounded vertices (corners). The electrode assembly 300 may include a foam or tape outline 304, a conductive anisotropic material layer 302, an electrode layer 306, a top cover 308, and an electrical power connection 301, which may be similar to the foam or tape outlines 104, 204, conductive anisotropic material layers 102, 202, electrode layers 106, 206, top covers 108, 208, and electrical power connections 101, 201, except for their shapes. When placed on a subject to apply an electric field to a subject, the foam or tape outline 304 may be closest to the subject, and the top cover 308 may be furthest from the subject. When viewed in a direction perpendicular to a face of the electrode assembly 300 facing the subject, the electrode assembly 300 may have a rounded triangular shape.

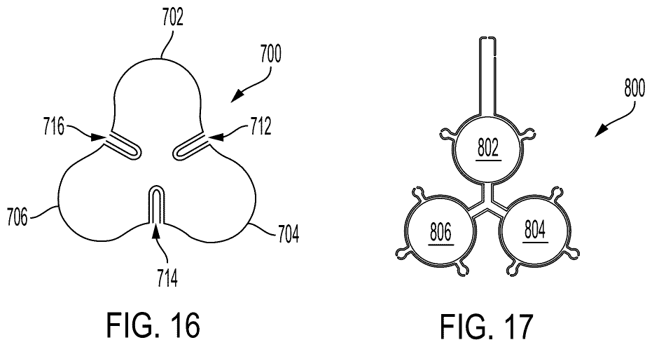

When viewed in a direction perpendicular to a front face 330 of the conductive anisotropic material layer 302, the conductive anisotropic material layer 302 may have a rounded triangular shape. The conductive anisotropic material layer 302 may include first edge 310, second edge 312, and third edge 314 with rounded vertices 316, 318, 320. In this way, first rounded vertex 316 may be disposed between first edge 310 and second edge 312; second rounded vertex 318 may be disposed between second edge 312 and third edge 314; and third rounded vertex 320 may be disposed between third edge 314 and first edge 310. In some embodiments, all three of the first edge 310, second edge 312, and third edge 314 may be relatively straight or have straight central portions, as in FIGS. 5 and 6. In some embodiments, all three of the first edge 310, second edge 312, and third edge 314 may be convex or bulging, such that the generally triangular shape has a more rounded shape. In some embodiments, all three of the first edge 310, second edge 312, and third edge 314 may be concave or indented, such that the generally triangular shape has a filled clover leaf shape, for example, as shown by the general shape of FIG. 16. In some embodiments, the generally triangular shape may be an equilateral triangle (as in FIGS. 5 and 6), an isosceles triangle, or a scalene triangle. In some embodiments, the three edges of the triangle may be of substantially equal length, thereby forming an equilateral rounded triangular configuration.

When viewed in a direction perpendicular to the front face 330 of the conductive anisotropic material layer 302, the shape of the foam or tape outline 304, the conductive anisotropic material layer 302, the electrode layer 306, and the top cover 308 may be similarly shaped. In some embodiments, the shape of the electrode layer 306 may correspond to the shape of the conductive anisotropic material layer 302 but may be a smaller version (for example, the electrode layer may be a single electrode element having a rounded triangular shape). As another example, and as shown in FIG. 6, the electrode layer 306 may include three circular portions 350, 352, and 354 arranged in a triangular pattern. As a part of the electrode assembly 300, the three circular portions 350, 352, and 354 of the electrode layer 306 may align with the rounded vertices 316, 318, and 320, respectively, of the conductive anisotropic material layer 302.

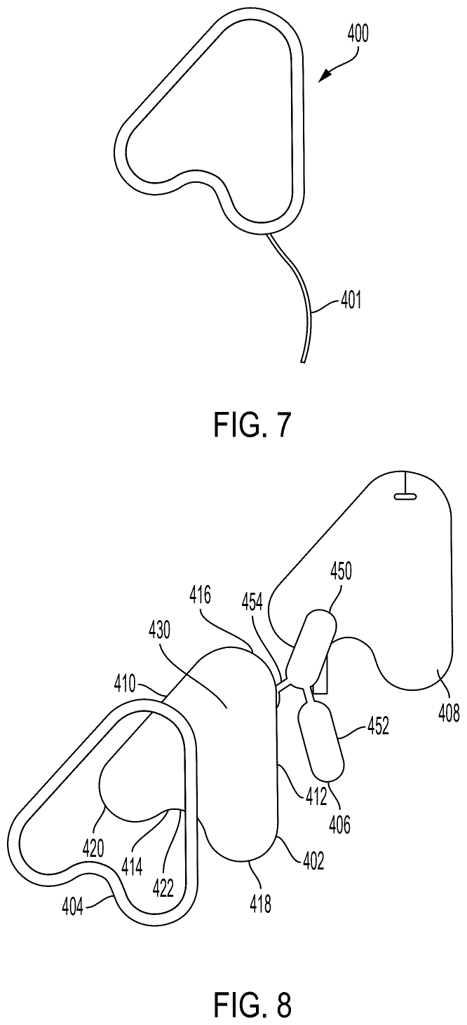

FIG. 7 depicts an example of an electrode assembly 400 having a rounded arrowhead shape, and FIG. 8 depicts an exploded view of the example of the electrode assembly 400 of FIG. 7. The electrode assembly 400 may be similar to the electrode assemblies 100, 200, and 300, but the electrode assembly 400 may have a rounded arrowhead shape. The electrode assembly 400 may include a foam or tape outline 404, a conductive anisotropic material layer 402, an electrode layer 406, a top cover 408, and an electrical power connection 401, which may be similar to the foam or tape outlines 104, 204, 304, conductive anisotropic material layers 102, 202, 302, electrode layers 106, 206, 306, top covers 108, 208, 308, and electrical power connections 101, 201, 301, except for their shapes. When placed on a subject to apply an electric field to a subject, the foam or tape outline 404 may be closest to the subject, and the top cover 408 may be furthest from the subject. When viewed in a direction perpendicular to a face of the electrode assembly 400 facing the subject, the electrode assembly 400 may have a rounded arrowhead shape.

When viewed in a direction perpendicular to a front face 430 of the conductive anisotropic material layer 402, the conductive anisotropic material layer 402 may have a rounded arrowhead shape. The conductive anisotropic material layer 402 may include first edge 410, second edge 412, and third edge 414 with rounded vertices 416, 418, and 420. The third edge 414 may include a concave portion or indentation 422, thereby completing the rounded arrowhead shape. In some embodiments, the rounded arrowhead shape may resemble an equilateral triangle, an isosceles triangle (as in FIGS. 7 and 8), or a scalene triangle.

When viewed in a direction perpendicular to the front face 430 of the conductive anisotropic material layer 402, the shape of the foam or tape outline 404, the conductive anisotropic material layer 402, the electrode layer 406, and the top cover 408 may be similarly shaped. In some embodiments, the shape of the electrode layer 406 may correspond to the shape of the conductive anisotropic material layer 402 but may be a smaller version (for example, the electrode layer may be a single electrode element having a rounded arrowhead shape). As another example, and as shown in FIG. 8, the electrode layer 406 may include three ovals or arms 450, 452, and 454 arranged in a triangular pattern (the view of 454 is obscured). As a part of the electrode assembly 400, the three ovals or arms 450, 452, and 454 of the electrode layer 406 may align with the rounded vertices 416, 418, and 420, respectively, of the conductive anisotropic material layer 402. The three ovals or arms 450, 452, and 454 of the electrode layer 406 need not be parallel sided ovals as shown, but could also be, for example, one or more stretched asymmetric oval, ovaloid, ovoid, or ovate shapes, or one or more asymmetric oval, ovaloid, ovoid, or ovate shapes, or a combination thereof.

The rounded arrowhead shape (FIGS. 7-8) may be viewed as an adaptation of the rounded triangular shape (FIGS. 5-6). In general, this embodiment is representative of an embodiment wherein the conductive anisotropic material layer has a generally rounded triangular shape with either one or two concave edges and/or one or two convex edges. For example, one convex edge and two concave edges results in a pear-shaped layer.

FIGS. 9A-9D depict electrode assemblies 100A, 100B, 100C, 100D in different sizes and configurations of the electrode assembly 100 having the stretched asymmetric oval or stretched ovaloid shape of FIG. 1, as may be required for different treatments or different body contours of different subjects.

FIGS. 10A-10D depict electrode assemblies 200A, 200B, 200C, 200D in different sizes of the electrode assembly 200 having the asymmetric oval, ovaloid or ovate shape of FIG. 3, as may be required for different treatments or different body contours of different subjects.

FIGS. 11A-11D depict electrode assemblies 300A, 300B, 300C, 300D in different sizes of the electrode assembly 300 having the rounded triangular shape of FIG. 5, as may be required for different treatments or different body contours of different subjects.

FIGS. 12A-12D depict electrode assemblies 400A, 400B, 400C, 400D in different sizes of the electrode assembly 400 having the rounded arrowhead shape of FIG. 7, as may be required for different treatments or different body contours of different subjects.

The sizes of the electrode assemblies discussed herein may vary depending on where the electrode assembly is placed on the subject, the subject's body size and shape, and/or on the size of the electric field to be generated using the electrode assembly. As an example, the area of each of the electrode assemblies 100, 200, 300, 400 may be in the range of from 5 cm² to 350 cm², such as, for example, approximately 100 cm². As an example, the area of each of the electrode assemblies 100A, 200A, 300A, 400A may be in the range of from 10 cm² to 40 cm² or 40 cm² to 70 cm², such as, for example, approximately 60 cm². As an example, the area of each of the electrode assemblies 100B, 200B, 300B, 400B may be in the range of from 70 cm² to 100 cm², such as, for example, approximately 90 cm². As an example, the area of each of the electrode assemblies 100C, 200C, 300C, 400C may be in the range of from 100 cm² to 130 cm², such as, for example, approximately 110 cm². As an example, the area of each of the electrode assemblies 100D, 200D, 300D, 400D may be in the range of from 130 cm² to 160 cm², such as, for example, approximately 140 cm². In other embodiments, the area of each of the electrode assemblies 100D, 200D, 300D, 400D may be in the range of from 160 cm² to 200 cm², such as, for example, approximately 180 cm²; in the range of from 200 cm² to 250 cm², such as, for example, approximately 220 cm²; in the range of from 250 cm² to 300 cm², such as, for example, approximately 260 cm². Similar sizes and size ranges may be applicable to any of the electrode assemblies discussed herein.

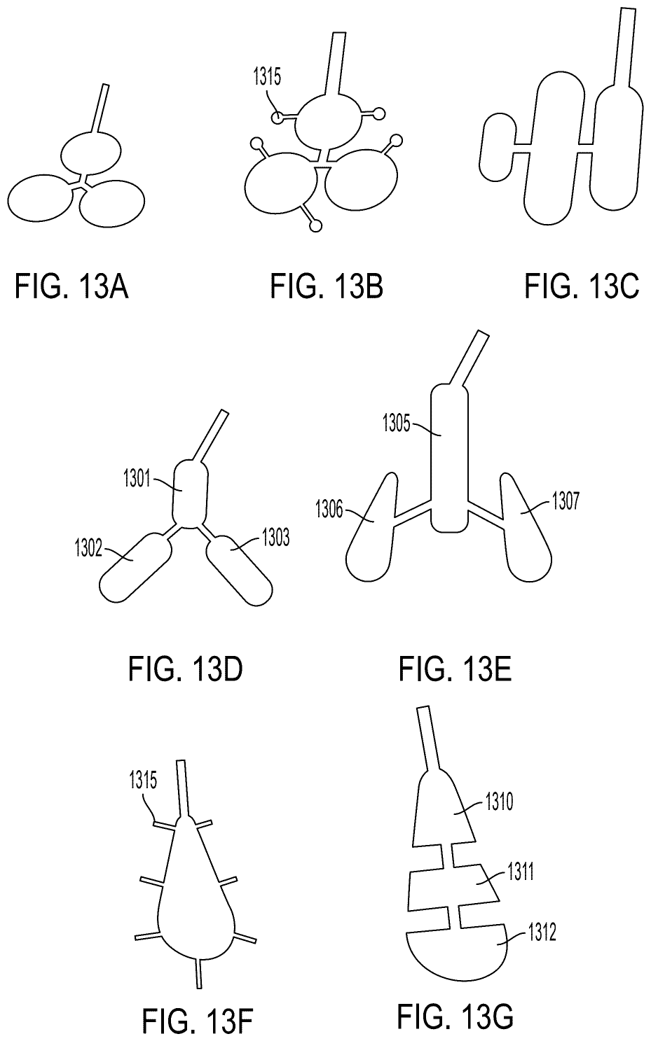

FIGS. 13A-13G depict different examples of electrode layers. The electrode layers may be used in various embodiments of the electrode assemblies 100, 200, 300, 400. As shown in FIGS. 13A-13E and 13G, each electrode layer may have three electrode layer lobes. Alternatively, as shown in FIG. 13F, an electrode layer may have only one electrode layer lobe. One having ordinary skill in the art will understand that any other number of electrode layer lobes may be used as needed; for example, an electrode layer may have one, two, three, four, five, or any other number of electrode layer lobes. As is also shown in FIGS. 13A-13G, the individual lobes of the electrode layer may be any shape and may, but need not, be the same size and/or shape. As shown in FIGS. 13A-13B, for example, each lobe may be a circular shape or a substantially circular shape. As shown in FIGS. 13C-13D, each lobe may be an ellipse shape or a stretched ellipse shape (also known as a “stadium” shape) of differing sizes or a same size. For example, in FIG. 13C, the three lobes are of different sizes. As another example, in FIG. 13D, lobes 1302 and 1303 are the same size and lobe 1301 is smaller and a different size than lobes 1302 and 1303. As shown in FIG. 13E, two lobes 1306 and 1307 may be stretched asymmetric ovals and one lobe 1305 may be a stretched ellipse shape. As shown in FIG. 13F, a single lobe may have a stretched asymmetric oval shape. And as shown in FIG. 13G, one lobe 1310 may have a triangular or substantially rounded triangular shape, a second lobe 1311 may have a isosceles trapezoid or substantially rounded isosceles trapezoidal shape, and a third lobe 1312 may have a half-circular or substantially rounded half-circular shape. As will be understood by one having ordinary skill in the art, the foregoing lobe shapes are exemplary and not limiting, and any other suitable lobe shape may be used. As shown in FIGS. 13B and 13F and discussed above, the electrode layer may include protrusions 1315 for positioning thermistors thereon, regardless of the shape(s) of the electrode lobe(s).

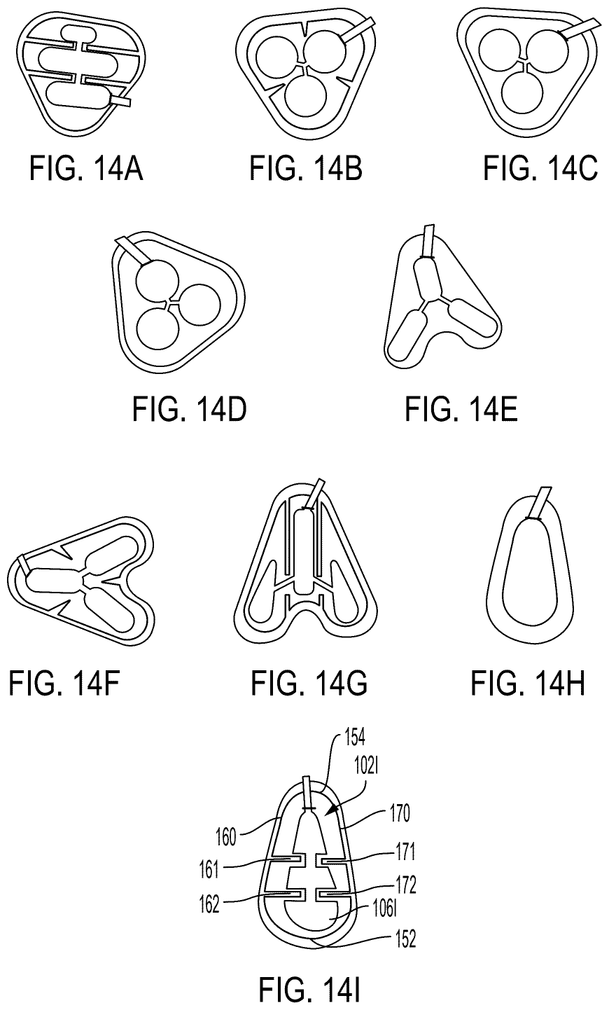

FIGS. 14A-14I depict examples of electrode assemblies. The view of the electrode assemblies is from the back face, instead of the front face, as in FIGS. 2-8. For clarity, the top cover is not depicted in the electrode assemblies. As one illustrative, non-limiting example, FIG. 14I shows that the conductive anisotropic material layer 1021 may include: first slits 161, 162 extending from a first edge 160 of the conductive anisotropic material layer 1021 toward a major axis of symmetry of the conductive anisotropic material layer 1021; and second slits 171, 172 extending from a second edge 170 of the conductive anisotropic material layer 1021 toward the major axis of symmetry of the conductive anisotropic material layer 1021. The first edge 160 may be opposite the second edge 170, and the first slits 161, 162, respectively, may be opposite the respective opposing second slits 171, 172 (slit 161 opposite slit 171, and slit 162 opposite slit 172, as shown in FIG. 141). In some embodiments, the top slits 161, 171 may have a length within 20% of a length of the bottom slits 162, 172. In another embodiment, the top slits 161, 171 may have a length that is equal to or within 5% of a length of the bottom slits 162, 172 (for example, equal to or within 10%, 15%, 20%, 25%, 30%, 35%, 40%, 45%, or 50% of a length of the bottom slits 162, 172, or any combination of range end points enumerated herein). In yet another embodiment, the top slits 161, 171 may have a length approximately 5%, 10%, 15%, 20%, 25%, 30%, 35%, 40%, 45%, or 50% less than a length of the bottom slits 162, 172. In some embodiments, the bottom slits 162, 172 may be closer to a bottom end 152 of the conductive anisotropic material layer 1021 than the top slits 161, 171 are to a top end 154. In some embodiments, the distance (or the average distance) of the bottom slits 162, 172 to the bottom end 152 may be less than the distance (or the average distance) of the top slits 161, 171 to the top end 154 of the conductive anisotropic material layer 1021. In some embodiments, for example as shown in FIGS. 14A-14B and 14F-14G, slits may be incorporated into the conductive anisotropic material layer of the electrode assembly regardless of the shape of the electrode layer and the individual electrode lobes. The slits in the conductive anisotropic material layer may have different shapes. For example, as shown in FIGS. 14A, 14G, and 14I, the slits in the conductive anisotropic material layer have a rectangular shape. For example, as shown in FIGS. 14B and 14F, the slits in the conductive anisotropic material layer have a triangular shape. In some embodiments, for example as shown in FIGS. 14C-14E and 14H, slits may not be utilized at all in the conductive anisotropic material layer of the electrode assembly. As viewed from the back face, some portion of the (larger area) conductive anisotropic material layer 1021 is obscured by the (smaller area) electrode layer 1061, which is closer to the back face of the electrode assembly 14I.

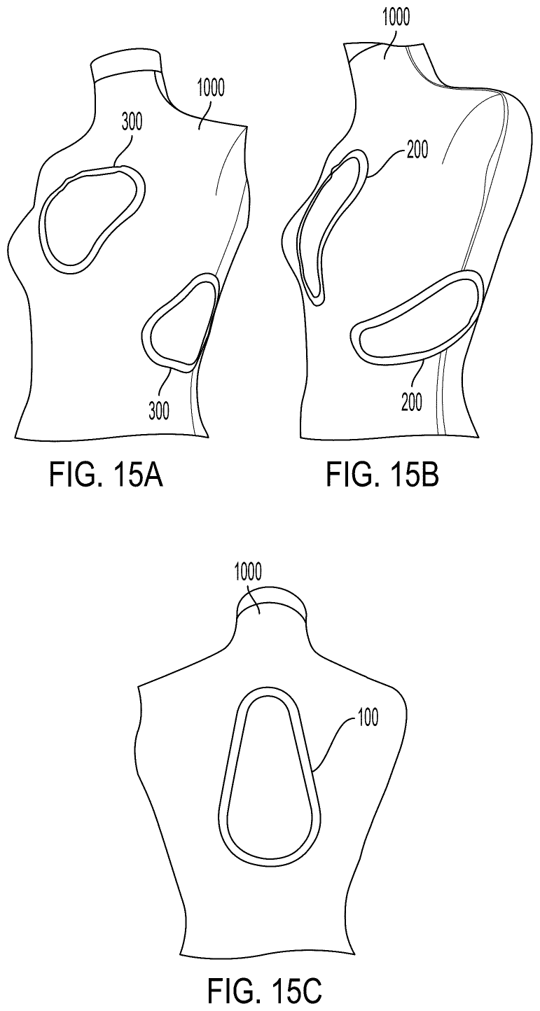

FIGS. 15A-15C depict positioning of electrode assemblies on a subject's body 1000 for application of TTFields. For clarity, the subject's body 1000 is depicted as a mannequin.

FIG. 15A depicts using two rounded triangular-shaped electrode assemblies 300 positioned on the subject's body 1000, particularly to the subject's (side) torso and chest. The shape of the electrode assemblies 300 may allow for close and comfortable fitting to the subject.

FIG. 15B depicts using two asymmetric oval, ovaloid, or ovate shaped electrode assemblies 200 positioned on the subject's body 1000, particularly to the subject's (side) torso and chest. The shape of the electrode assemblies 200 may allow for close and comfortable fitting to the subject.

FIG. 15C depicts using a stretched asymmetric oval, ovaloid, or ovate shaped electrode assembly 100 positioned on the subject's body 1000, particularly to the subject's back, between the shoulder blades. The shape of the electrode assemblies 100 may allow for close and comfortable fitting to the subject.

FIG. 16 depicts an example of a conductive anisotropic layer 700. The conductive anisotropic layer 700 may include three circular portions 702, 704, 706 in a triangular formation. The conductive anisotropic layer 700 may be viewed as being similar to the conductive anisotropic layer 302, but each edge of the triangle includes a concave portion or indentation, such as the concave portion or indentation 422 of the conductive anisotropic layer 402 (FIG. 8). The conductive anisotropic layer 700 may include slits 712, 714, 716 between the circular portions 702, 704, 706. The slits 712, 714, 716 provide an extra degree of flexibility to the conductive anisotropic layer 700 and therefore may provide extra comfort to the subject when wearing an electrode assembly having the conductive anisotropic layer 700, especially when the electrode assembly covers a non-planar surface. The conductive anisotropic layer 700 may otherwise be similar to the other conductive anisotropic layers described herein.

FIG. 17 depicts an example of an electrode layer 800. The electrode layer 800 may include three circular portions 802, 804, 806 in a triangular pattern. The electrode layer 800 may be similar to the electrode layer 306 (FIG. 6). The electrode layer 800 may otherwise be similar to the other electrode layers described herein. The electrode layer 800 may be used in combination with the conductive anisotropic layer 700 (FIG. 16) in an electrode assembly.

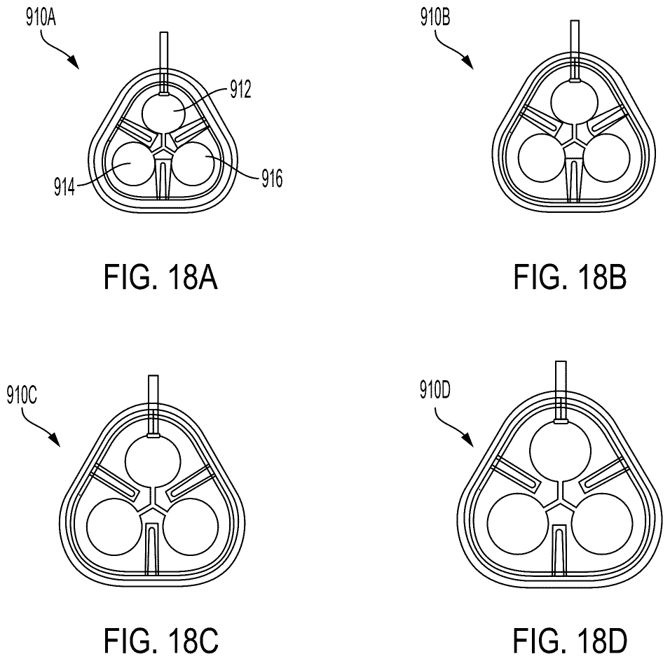

FIGS. 18A-18D depict different sizes of an electrode assembly. Electrode assemblies 910A, 910B, 910C, 910D are different sizes and configurations of an electrode assembly having a conductive anisotropic layer similar to the conductive anisotropic layer 302 (FIG. 6) and an electrode layer similar to the electrode layer 800 (FIG. 17). The electrode layer may include a three-lobe pattern with circular portions 912, 914, 916 in a triangular pattern.

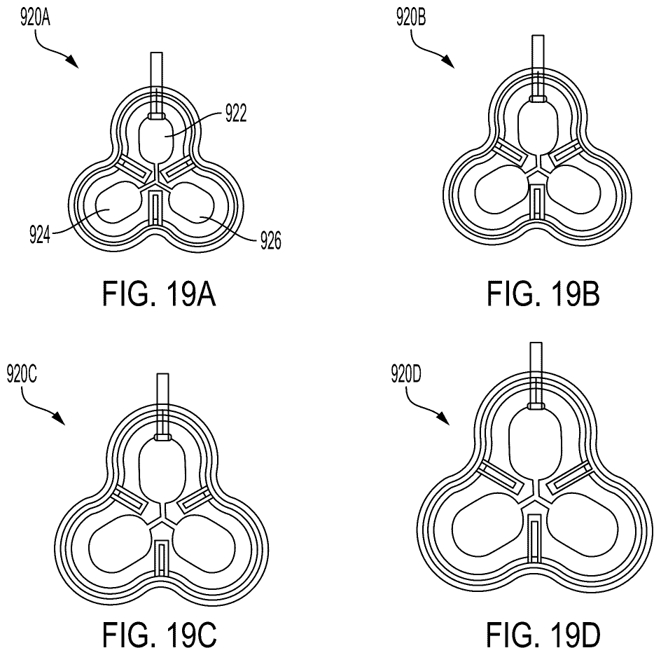

FIGS. 19A-19D depict different sizes of an electrode assembly. Electrode assemblies 920A, 920B, 920C, 920D are different sizes and configurations of an electrode assembly having a conductive anisotropic layer similar to the conductive anisotropic layer 700 (FIG. 16) and an electrode layer similar to the electrode layer in FIG. 13D. The electrode layer may include a three-lobe pattern with oval portions 922, 924, 926 in a triangular pattern.

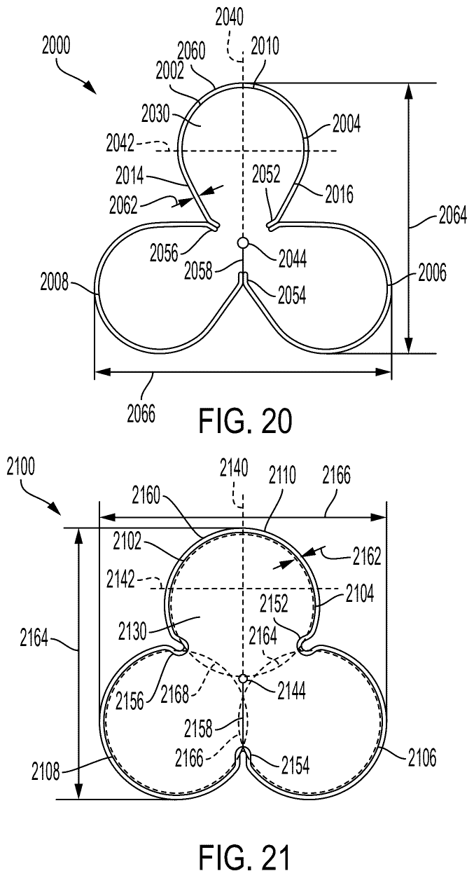

FIG. 20 depicts an example of an electrode assembly 2000, and FIG. 21 depicts an example of an electrode assembly 2100. The electrode assembly 2000, 2100 may include an electrode layer 2002, 2102 and a top cover 2060, 2160. In some embodiments, the electrode assembly 2000, 2100 does not include a layer of conductive anisotropic material. In some embodiments, the electrode layer 2002, 2102 may include a foam or tape outline, similar to other embodiments discussed herein. The electrode layer 2002, 2102 may include a front face 2030, 2130 adapted to face toward the subject. When viewed in a direction perpendicular to the front face 2030, 2130, the electrode layer 2002, 2102 may include three lobes or portions 2004, 2006, 2008, and 2104, 2106, 2108 in a triangular formation. In some embodiments, the triangular formation of the lobes 2004, 2006, 2008, and the lobes 2104, 2106, 2108 may be an equilateral triangle or an approximately equilateral triangle (as in FIG. 20 and FIG. 21), an isosceles triangle, or a scalene triangle.

Referring to FIG. 20, each lobe 2004, 2006, 2008 may have a stretched asymmetric oval shape, similar to the stretched asymmetric oval shape of the electrode assembly 100 (FIG. 1). In some embodiments, each lobe 2004, 2006, 2008 may have an asymmetric oval, ovaloid, ovoid, or ovate shape or a stretched asymmetric oval, stretched, stretched, or ovate shape. As part of the stretched asymmetric oval shape, each lobe 2004, 2006, 2008 may have a first end 2010 and edges 2014, 2016 extending from the first end to an imaginary second end. The first end 2010 may have a first radius of curvature, and the imaginary second end may be opposite the first end 2010 and have a second radius of curvature. The first radius of curvature of the first end 2010 may be greater than the second radius of curvature of the imaginary second end. Each lobe 2004, 2006, 2008 may have a mirror symmetry configuration about a major axis of symmetry 2040 and an asymmetric configuration about a minor axis of symmetry 2042 perpendicular to the major axis. The major axis of symmetry 2040 may pass through a center 2044 of the electrode layer 2002. For clarity, references 2010, 2014, 2016, 2040, 2042 are not depicted for lobes 2006, 2008.

Referring to FIG. 21, each lobe 2104, 2106, 2108 may have a partial circular shape. In some embodiments, the partial circular shape may be greater than at least a semicircle and less than at most a circle. As part of the circular shape, each lobe 2104, 2106, 2108 may have a first end 2110. The first end 2110 of each lobe 2104, 2106, 2108 may have a circular shape with a same radius of curvature. The circular shape of the first end 2110 of each lobe 2104, 2106, 2108 may not be a complete circle, and a perimeter of the first end 2110 of each lobe 2104, 2106, 2108 may be 50% or more in the shape of a circle. As an example, the perimeter of the first end 2110 of each lobe 2104, 2106, 2108 may be 50%, 60%, 70%, 80%, or 90% in the shape of a circle. Each lobe 2104, 2106, 2108 may have a mirror symmetry configuration about a major axis of symmetry 2140 and an asymmetric configuration about a minor axis of symmetry 2142 perpendicular to the major axis. The major axis of symmetry 2140 may pass through a center 2144 of the electrode layer 2102. For clarity, references 2110, 2140, 2142 are not depicted for lobes 2006, 2008.

In some embodiments, as an aspect of the partial circular shape of the perimeter of each lobe 2104, 2106, 2108, each lobe 2104, 2106, 2108 may be represented by an imaginary circle 2164, 2166, 2168. In some embodiments, the electrode layer 2102 does not include a point that is overlapped by all three of the three imaginary circles 2164, 2166, 2168. In some embodiments, the center 2144 may be outside of each of the three imaginary circles 2164, 2166, 2168. In some embodiments, the electrode layer 2102 includes at least one point that is overlapped by the three imaginary circles 2164, 2166, 2168. In some embodiments, the center 2144 may be inside each of the three imaginary circles 2164, 2166, 2168. In some embodiments, the center 2144 may be equidistant from each of the three imaginary circles 2164, 2166, 2168.

Referring back to FIG. 20 and FIG. 21, to assist with the comfort of the subject when wearing the electrode assembly 2000, 2100 having the electrode layer 2002, 2102, the electrode layer 2002, 2102 may include one or more indentations, concave portions, or slits between the lobes. In some embodiments, the electrode layer 2002, 2012 may include an indentation, a concave portion, or a slit (2052, 2054, 2056, or 2152, 2154, 2156) between at least two of the lobes. In some embodiments, the electrode layer 2002, 2012 may include indentations or concave portions 2052, 2054, 2056, 2152, 2154, 2156 (i.e., in the three locations between adjacent lobes for the three lobes of each electrode assembly 2000, 2100). For the electrode layer 2002, indentation 2052 may be between lobes 2004, 2006; indentation 2054 may be between lobes 2006, 2008; and indentation 2056 may be between lobes 2004, 2008. For the electrode layer 2102, indentation 2152 may be between lobes 2104, 2106; indentation 2154 may be between lobes 2106, 2108; and indentation 2156 may be between lobes 2104, 2108. In some embodiments, the electrode layer 2002, 2012 may include a slit 2058, 2158 extending from indentation 2054, 2154, respectively, to the center 2044, 2144 or toward the center 2044, 2144. The electrode layer 2002, 2102 may otherwise be similar to the other electrode layers described herein.

In some embodiments, the electrode assembly 2000, 2100 may include the top cover 2060, 2160. When viewed in a direction perpendicular to the front face 2030, 2130, the top cover 2060, 2160 may have the same shape as the electrode layer 2002, 2102 but may be slightly larger. In some embodiments, a distance 2062, 2162 between a perimeter of the top cover 2060, 2160 and the perimeter of the electrode layer may be greater than at least 1 mm and less than at most 20 mm. In some embodiments, the distance 2062, 2162 may be 1 mm, 3 mm, 5 mm, 7 mm, 9 mm, 10 mm, 15 mm, or 20 mm.

The electrode assembly 2000, 2100 may be provided in one or more sizes, as discussed herein. In some embodiments, a length 2064, 2164 of the electrode assembly 2000, 2100 may be greater than at least 5 cm and less than at most 30 cm. In some embodiments, the length 2064, 2164 may be 5 cm, 10 cm, 15 cm, 20 cm, 25 cm, or 30 cm. In some embodiments, a width 2066, 2166 of the electrode assembly 2000, 2100 may be greater than at least 5 cm and less than at most 30 cm. In some embodiments, the width 2066, 2166 may be 5 cm, 10 cm, 15 cm, 20 cm, 25 cm, or 30 cm. In some embodiments, the length 2064, 2164 and the width 2066, 2166 are the same. In some embodiments, the length 2064, 2164 and the width 2066, 2166 are not the same. In some embodiments, an area of the electrode assembly 2000, 2100 may be greater than at least 8 cm² and less than at most 300 cm². In some embodiments, the area of the electrode assembly 2000, 2100 may be 8 cm², 10 cm², 20 cm², 30 cm², 40 cm², 50 cm², 60 cm², 70 cm², 80 cm², 90 cm², 100 cm², 120 cm², 140 cm², 160 cm², 180 cm², 200 cm², 220 cm², 240 cm², 260 cm², 280 cm², or 300 cm², or range in size between any two of the sizes listed here.

In some embodiments, the electrode assembly 2000, 2100 may include a layer of conductive anisotropic material. In such embodiments having a layer of conductive anisotropic material, when viewed in a direction perpendicular to the front face 2030, 2130, the electrode assembly, the layer of conductive anisotropic material, and the top cover may have a rounded triangular shape (e.g., as in the electrode assembly 300 in FIGS. 5 and 6, the electrode assembly 300A, 300B, 300C, 300D in FIGS. 11A-11D, or the electrode assembly in FIGS. 14A-14D). Alternatively, the electrode assembly, the layer of conductive anisotropic material, and the top cover may have a contoured or indented rounded triangular shape (e.g., as in the electrode assembly 920A-D in FIGS. 19A-D), with or without slits.

As such, different and various combinations of the exemplary conductive anisotropic layers described herein and the exemplary electrode layers described herein may be used together in an electrode assembly.

The embodiments described herein may incorporate into the electrode assembly a conductive anisotropic material layer (for example, 102, 202, 302, 402) which may be a sheet of material having anisotropic thermal properties and/or anisotropic electrical properties, as described below. If the sheet of material has anisotropic thermal properties, then the sheet spreads the heat out more evenly over a larger surface area. If the sheet of material has anisotropic electrical properties, then the sheet spreads the current out more evenly over a larger surface area. In each case, this may lower the temperature of hot spots and may raise the temperature of the cooler regions when a given AC voltage is applied to the electrode assembly (as compared to electrode assemblies lacking such sheet of anisotropic material). Accordingly, the current can be increased (thereby increasing the therapeutic effect) without exceeding the safety temperature threshold at any point on the subject's skin.

In some embodiments, the anisotropic material may be anisotropic with respect to electrical conductivity properties. In some embodiments, the anisotropic material may be anisotropic with respect to thermal conductivity properties. In some preferred embodiments, the anisotropic material may be anisotropic with respect to both electrical conductivity properties and thermal conductivity properties.

The anisotropic thermal properties of the anisotropic material may include directional thermal properties. As an example, the sheet of anisotropic material may have a first thermal conductivity in a direction that is perpendicular to its front face. As an example, the thermal conductivity of the sheet in directions parallel to the front face may be more than two times higher than the first thermal conductivity. In some embodiments, the thermal conductivity in the parallel directions may be more than ten times higher than the first thermal conductivity. For example, the thermal conductivity of the sheet in directions that are parallel to the front face may be more than: 1.5 times, 2 times, 3 times, 5 times, 10 times, 20 times, 30 times, 100 times, 200 times, or even more than 1,000 times higher than the first thermal conductivity. In some embodiments, the thermal conductivity of the sheet of anisotropic material in directions that are parallel to the front face may be between 5 times and 30 times higher than the first thermal conductivity. For example, the thermal conductivity of a sheet of pyrolytic graphite in directions that are in the x-y plane may be between 10 times and 20 times higher than its thermal conductivity in the perpendicular z-direction.

The anisotropic electrical properties of the anisotropic material may include directional electrical properties. In some embodiments, the sheet may have a first resistance in a direction that is perpendicular to its front face. In some embodiments, resistance of the sheet in directions parallel to the front face may be less than the first resistance. In some embodiments, the resistance in the parallel directions may be less than half of the first resistance or less than 10% of the first resistance. For example, the resistance of the sheet 102, 202, 302, 402 in directions that are parallel to the front face may be less than: 75%, 50%, 40%, 30%, 20%, 10%, 5%, 1%, 0.5%, 0.1%, or even less than 0.05% of the first resistance. In some embodiments, the resistance of the sheet of anisotropic material in directions that are parallel to the front face may be between 0.05% and 10% of the first resistance. For example, the electrical resistivity of the anisotropic material (e.g., a sheet of pyrolytic graphite) in directions that are in the x-y plane may be approximately three orders of magnitude (1,000 times) lower than its electrical resistivity in the perpendicular z-direction.

In some aspects, the conductive anisotropic material layer can be or can comprise a synthetic graphite. In additional aspects, the conductive anisotropic material layer can be or can comprise a layer of pyrolytic graphite, graphitized polymer film, or graphite foil made from compressed high purity exfoliated mineral graphite.

In some embodiments (e.g., when the conductive anisotropic material layer is a sheet of pyrolytic graphite), the conductive anisotropic material layer has both anisotropic electrical properties and anisotropic thermal properties.

### Illustrative Embodiments

The invention includes other illustrative embodiments (“Embodiments”) as follows.

Illustrative Embodiment 1: An electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

Illustrative Embodiment 2: The electrode assembly of Illustrative Embodiment 1, wherein, when viewed in the direction perpendicular to the front face of the conductive anisotropic material layer, the conductive anisotropic material layer has mirror symmetry about a major axis and is asymmetric with respect to mirror symmetry about a minor axis perpendicular to the major axis.

Illustrative Embodiment 3: The electrode assembly of Illustrative Embodiment 1, wherein, when viewed in the direction perpendicular to the front face of the conductive anisotropic material layer, the conductive anisotropic material layer includes edges extending between the first end and the second end, at least one of the edges including at least a portion which is substantially straight.

Illustrative Embodiment 4: The electrode assembly of Illustrative Embodiment 1, wherein, when viewed in the direction perpendicular to the front face of the conductive anisotropic material layer, the conductive anisotropic material layer includes edges extending between the first end and the second end, at least one of the edges including at least a portion having a convex or bulging shape.

Illustrative Embodiment 5: The electrode assembly of Illustrative Embodiment 1, wherein, when viewed in the direction perpendicular to the front face of the conductive anisotropic material layer, the electrode layer has a shape similar to the asymmetric oval, ovaloid, ovoid, or ovate shape or stretched oval, ovaloid, ovoid, or ovate shape of the conductive anisotropic material layer.

Illustrative Embodiment 6: The electrode assembly of Illustrative Embodiment 1, wherein, when viewed in the direction perpendicular to the front face of the conductive anisotropic material layer, an outer perimeter of the electrode layer has an asymmetric oval, ovaloid, ovoid, or ovate shape or stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature, wherein the first end and the second end of the electrode layer corresponds to the first end and the second end, respectively, of the conductive anisotropic material layer, and wherein the first radius of curvature of the electrode layer is smaller than the first radius of curvature of the conductive anisotropic material layer.

Illustrative Embodiment 7: The electrode assembly of Illustrative Embodiment 1, wherein, when viewed in the direction perpendicular to the front face of the conductive anisotropic material layer, a length of the conductive anisotropic material layer along a major axis of symmetry is about 10 cm to about 45 cm.

Illustrative Embodiment 8: The electrode assembly of Illustrative Embodiment 1, wherein, when viewed in the direction perpendicular to the front face of the conductive anisotropic material layer, an area enclosed by a perimeter of the conductive anisotropic material layer is at least 10% larger than an area enclosed by a perimeter of the electrode layer.

Illustrative Embodiment 9: The electrode assembly of Illustrative Embodiment 1, wherein, when viewed in the direction perpendicular to the front face of the conductive anisotropic material layer, the conductive anisotropic material layer comprises: a first slit extending from a first edge of the conductive anisotropic material layer toward a major axis of symmetry of the conductive anisotropic material layer; and a second slit extending from a second edge of the conductive anisotropic material layer toward the major axis of symmetry of the conductive anisotropic material layer, wherein the first edge is opposite the second edge, wherein the first slit is opposite the second slit, and wherein the first slit has a length equal to or within 20% of a length of the second slit.

Illustrative Embodiment 10: The electrode assembly of Illustrative Embodiment 9, wherein the first slit and the second slit are both closer to the first end than the second end of the conductive anisotropic material layer.

Illustrative Embodiment 11: The electrode assembly of Illustrative Embodiment 1, wherein the conductive anisotropic material layer is sandwiched between a first conductive adhesive or hydrogel layer and a second conductive adhesive or hydrogel layer, and wherein the conductive anisotropic material layer is or comprises a sheet of graphite.

Illustrative Embodiment 11A: The electrode assembly of Illustrative Embodiment 1, wherein the conductive anisotropic material is or comprises a sheet of graphite.

Illustrative Embodiment 12: The electrode assembly of Illustrative Embodiment 1, wherein the electrode assembly is adapted to be located on a back of the subject between shoulder blades of the subject.

Illustrative Embodiment 12A: The electrode assembly of Illustrative Embodiment 1, wherein the electrode assembly is adapted to be located on a back of the subject over a shoulder blade of the subject.

Illustrative Embodiment 12B: The electrode assembly of Illustrative Embodiment 1, wherein the electrode assembly is adapted to deliver tumor treating fields to the subject.

Illustrative Embodiment 13: An electrode assembly for delivering an alternating electric field to a subject, the electrode assembly comprising: an electrode layer having a front face and a back face, the front face of the electrode layer adapted to face toward the subject, the back face being opposite to the front face, wherein, when viewed in a direction perpendicular to the front face of the electrode layer, the electrode layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

Illustrative Embodiment 14: The electrode assembly of Illustrative Embodiment 13, wherein the electrode layer has a single electrode element having the asymmetric oval, ovaloid, ovoid, or ovate shape or stretched asymmetric oval, ovaloid, ovoid, or ovate shape of the electrode layer.

Illustrative Embodiment 15: The electrode assembly of Illustrative Embodiment 13, wherein the electrode layer comprises a first electrode element, a second electrode element, and a third electrode element, wherein, when viewed in a direction perpendicular to the front face of the conductive anisotropic material layer, the first electrode element has a substantially rounded triangular shape, the second electrode element has a substantially rounded isosceles trapezoidal shape, and the third electrode element has a substantially rounded half-circular shape.

Illustrative Embodiment 16: The electrode assembly of Illustrative Embodiment 13, further comprising a conductive anisotropic material layer electrically coupled to the electrode layer, the conductive anisotropic material layer having a front face and a back face, the back face of the conductive anisotropic material layer facing toward the front face of the electrode layer, the front face of the conductive anisotropic material layer adapted to face toward the subject.

Illustrative Embodiment 17: An electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer comprises: a first edge, a second edge, and a third edge in a triangular configuration; a first rounded vertex between the first edge and second edge, a second rounded vertex between the second edge and third edge; and a third rounded vertex between the third edge and first edge.

Illustrative Embodiment 18: The electrode assembly of Illustrative Embodiment 17, wherein the first edge, the second edge, and the third edge are of substantially equal length, thereby forming an equilateral rounded triangular configuration.

Illustrative Embodiment 18A: The electrode assembly of Illustrative Embodiment 17, wherein each of the first edge, the second edge, and the third edge include at least a portion which is substantially straight.

Illustrative Embodiment 18B: The electrode assembly of Illustrative Embodiment 17, wherein each of the first edge, the second edge, and the third edge includes at least a portion having a bulging, convex shape.

Illustrative Embodiment 18C: The electrode assembly of Illustrative Embodiment 17, wherein, when viewed in the direction perpendicular to the front face of the conductive anisotropic material layer, one or more electrode elements of the electrode layer have a circular shape.

Illustrative Embodiment 18D: The electrode assembly of Illustrative Embodiment 17, wherein, when viewed in the direction perpendicular to the front face of the conductive anisotropic material layer, one or more electrode elements of the electrode layer have an oval, ovaloid, ovoid, or ovate shape, or a stretched oval, ovaloid, ovoid, or ovate shape, or an asymmetric oval, ovaloid, ovoid, or ovate shape, or an asymmetric stretched oval, ovaloid, ovoid, or ovate shape.

Illustrative Embodiment 18E: The electrode assembly of Illustrative Embodiment 17, wherein, when viewed in the direction perpendicular to the front face of the conductive anisotropic material layer, the electrode layer comprises three circular-shaped electrode elements.

Illustrative Embodiment 18F: The electrode assembly of Illustrative Embodiment 17, wherein, when viewed in the direction perpendicular to the front face of the conductive anisotropic material layer, the electrode layer comprises three oval-, ovoid-, or ovate-shaped electrode elements, or three stretched oval-, ovoid-, or ovate-shaped electrode elements, or three asymmetric oval-, ovoid-, or ovate-shaped electrode elements, or three stretched asymmetric oval-, ovoid-, or ovate-shaped electrode elements.

Illustrative Embodiment 18G: The electrode assembly of Illustrative Embodiment 17, wherein the conductive anisotropic material layer is sandwiched between a first conductive adhesive or hydrogel layer and a second conductive adhesive or hydrogel layer, and wherein the conductive anisotropic material layer is or comprises a sheet of graphite.

Illustrative Embodiment 18H: The electrode assembly of Illustrative Embodiment 17, wherein, when viewed in the direction perpendicular to the front face of the conductive anisotropic material layer, one or more electrode elements of the electrode layer have a circular shape, or an oval, ovaloid, ovoid, or ovate shape, or a stretched oval, ovaloid, ovoid, or ovate shape, or an asymmetric oval, ovaloid, ovoid, or ovate shape, or a stretched asymmetric oval, ovaloid, ovoid, or ovate shape.

Illustrative Embodiment 19: The electrode assembly of Illustrative Embodiment 17, wherein, when viewed in the direction perpendicular to the front face of the conductive anisotropic material layer, the electrode layer comprises: a first circular-shaped or oval-, ovaloid-, ovoid-, or ovate-shaped, or stretched oval-, ovaloid-, ovoid-, or ovate-shaped, or asymmetric oval-, ovaloid-, ovoid-, or ovate-shaped, or stretched asymmetric oval-, ovaloid-, ovoid-, or ovate-shaped electrode element aligned with the first rounded vertex of the conductive anisotropic material layer; a second circular-shaped or oval-, ovaloid-, ovoid-, or ovate-shaped or stretched oval-, ovaloid-, ovoid-, or ovate-shaped, or asymmetric oval-, ovaloid-, ovoid-, or ovate-shaped, or stretched asymmetric oval-, ovaloid-, ovoid-, or ovate-shaped electrode element aligned with the second rounded vertex of the conductive anisotropic material layer; and a third circular-shaped or oval-, ovaloid-, ovoid-, or ovate-shaped, or stretched oval-, ovaloid-, ovoid-, or ovate-shaped, or asymmetric oval-, ovaloid-, ovoid-, or ovate-shaped, or, or stretched asymmetric oval-, ovaloid-, ovoid-, or ovate-shaped electrode element aligned with the third rounded vertex of the conductive anisotropic material layer.

Illustrative Embodiment 19A: The electrode assembly of Illustrative Embodiment 17, wherein, when viewed in the direction perpendicular to the front face of the conductive anisotropic material layer, the electrode layer comprises: a first stretched oval-, ovaloid-, ovoid-, or ovate-shaped electrode element aligned with the first rounded vertex of the conductive anisotropic material layer; a second stretched oval-, ovaloid-ovoid-, or ovate-shaped electrode element aligned with the second rounded vertex of the conductive anisotropic material layer; and a third stretched oval-, ovaloid-ovoid-, or ovate-shaped electrode element aligned with the third rounded vertex of the conductive anisotropic material layer.

Illustrative Embodiment 20: The electrode assembly of Illustrative Embodiment 17, wherein, when viewed in the direction perpendicular to the front face of the conductive anisotropic material layer, the conductive anisotropic material layer has mirror symmetry about a major axis and comprises: a first slit extending from the first edge of the conductive anisotropic material layer toward the major axis of the conductive anisotropic material layer; and a second slit extending from the second edge of the conductive anisotropic material layer toward the major axis of the conductive anisotropic material layer, wherein the first slit has a length equal to or within 20% of a length of the second slit.

Illustrative Embodiment 20A: The electrode assembly of Illustrative Embodiment 20, wherein, when viewed in the direction perpendicular to the front face of the conductive anisotropic material layer, the first slit is closer to the third edge than the second edge, and the second slit is closer to the third edge than the first edge.

Illustrative Embodiment 20B: The electrode assembly of Illustrative Embodiment 20, wherein at least one edge of the first edge, the second edge, or the third edge has a concave portion.

Illustrative Embodiment 21: The electrode assembly of Illustrative Embodiment 17, wherein the electrode assembly is adapted to be located on a front of the subject between pectoral areas of the subject.

Illustrative Embodiment 21A: The electrode assembly of Illustrative Embodiment 17, wherein the electrode assembly is adapted to be located on a front of the subject over a pectoral area of the subject.

Illustrative Embodiment 21B: The electrode assembly of Illustrative Embodiment 17, wherein the electrode assembly is adapted to be located on a side of a torso of the subject.

Illustrative Embodiment 21C: The electrode assembly of Illustrative Embodiment 17, wherein the electrode assembly is adapted to deliver tumor treating fields to the subject.

Illustrative Embodiment 21D: An electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer has a first edge, a second edge and a third edge in generally triangular configuration with rounded vertices, wherein at least one edge of the first edge, the second edge, and the third edge has a concave portion.

Illustrative Embodiment 21E: An electrode assembly for delivering an alternating electric field to a subject, the electrode assembly comprising: an electrode layer; and a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject, wherein, when viewed in a direction perpendicular to the front face of the conductive anisotropic material layer, the conductive anisotropic material layer has a first edge, a second edge and a third edge in generally triangular configuration with rounded vertices, wherein the third edge has a concave portion, thereby forming a rounded arrowhead-type shape.

Illustrative Embodiment 21F: The electrode assembly of Illustrative Embodiment 21E, wherein the first edge is longer than the third edge, and wherein the second edge is longer than the third edge.

Illustrative Embodiment 21G: The electrode assembly of Illustrative Embodiment 21E, wherein the generally triangular configuration is a generally isosceles triangular configuration, wherein the third edge is a shorter edge in the generally isosceles triangular configuration compared to the first edge and the second edge.

Illustrative Embodiment 21H: The electrode assembly of Illustrative Embodiment 21E, wherein two edges of the first edge, the second edge and the third edge have a concave portion.

Illustrative Embodiment 211: The electrode assembly of Illustrative Embodiment 21H, wherein two edges of the first edge, the second edge and the third edge have a concave portion and the conductive anisotropic material layer has a pear shape.

Illustrative Embodiment 21J: The electrode assembly of Illustrative Embodiment 21E, wherein the first edge, the second edge and the third edge each have a concave portion.

Illustrative Embodiment 22: An electrode assembly for delivering an alternating electric field to a subject, the electrode assembly comprising: an electrode layer having a front face and a back face, the front face of the electrode layer adapted to face toward the subject, the back face being opposite to the front face, wherein, when viewed in a direction perpendicular to the front face of the electrode layer, the electrode layer has three lobes in a triangular formation, wherein each lobe has an asymmetric oval, ovaloid, ovoid, or ovate shape, a stretched asymmetric oval, stretched ovaloid, stretched ovoid, or stretched ovate shape, or a stretched partial circular shape.

Illustrative Embodiment 22A: The electrode assembly of Illustrative Embodiment 22, wherein for each lobe having an asymmetric oval, ovaloid, ovoid, or ovate shape or a stretched asymmetric oval, stretched ovaloid, stretched ovoid, or stretched ovate shape, each lobe comprises a first end radially distant from a centroid of the electrode assembly and an imaginary second end radially closer to the centroid of the electrode assembly, the first end having a first radius of curvature, the second imaginary end being opposite the first end and having a second radius of curvature, the first radius of curvature being greater than the second radius of curvature.

Illustrative Embodiment 22B: The electrode assembly of Illustrative Embodiment 22, wherein for each lobe having a partial circular shape, the partial circular shape is greater than at least a semicircle and less than at most a circle.

Illustrative Embodiment 22C: The electrode assembly of Illustrative Embodiment 22, wherein for each lobe having a partial circular shape, a perimeter of each lobe is represented by an imaginary circle, and the electrode layer does not include a point that is overlapped by the three imaginary circles.

Illustrative Embodiment 22D: The electrode assembly of Illustrative Embodiment 22, wherein for each lobe having a partial circular shape, a perimeter of each lobe is represented by an imaginary circle, and the electrode layer includes a point that is overlapped by the three imaginary circles.

Illustrative Embodiment 22E: The electrode assembly of Illustrative Embodiment 22, wherein the electrode layer includes an indentation, a concave portion, or a slit between at least two of the lobes.

Illustrative Embodiment 22F: The electrode assembly of Illustrative Embodiment 22, wherein the electrode assembly does not include a layer of conductive anisotropic material.

Illustrative Embodiment 22G: The electrode assembly of Illustrative Embodiment 22, wherein the electrode assembly further comprises a layer of conductive anisotropic material.

Illustrative Embodiment 22H: The electrode assembly of Illustrative Embodiment 22G, wherein the layer of conductive anisotropic material has a rounded triangular shape.

Illustrative Embodiment 22I: The electrode assembly of Illustrative Embodiment 22G, wherein the layer of conductive anisotropic material has a rounded triangular shape with one or more contoured or indented edges.

Illustrative Embodiment 23: A method, machine, article of manufacture, and/or system substantially as shown and described.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. An electrode assembly for delivering an alternating electric field to a subject, the electrode assembly comprising:
an electrode layer; and
a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject,
wherein, when viewed in a direction perpendicular to the front face of the conductive anisotropic material layer, the conductive anisotropic material layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

2. The electrode assembly of claim 1, wherein, when viewed in the direction perpendicular to the front face of the conductive anisotropic material layer, the conductive anisotropic material layer has mirror symmetry about a major axis and is asymmetric with respect to mirror symmetry about a minor axis perpendicular to the major axis.

3. The electrode assembly of claim 1, wherein, when viewed in the direction perpendicular to the front face of the conductive anisotropic material layer, the conductive anisotropic material layer includes edges extending between the first end and the second end, at least one of the edges including at least a portion which is substantially straight.

4. The electrode assembly of claim 1, wherein, when viewed in the direction perpendicular to the front face of the conductive anisotropic material layer, the electrode layer has a shape similar to the asymmetric oval, ovaloid, ovoid, or ovate shape or stretched asymmetric oval, ovaloid, ovoid, or ovate shape of the conductive anisotropic material layer.

5. The electrode assembly of claim 1, wherein, when viewed in the direction perpendicular to the front face of the conductive anisotropic material layer, a length of the conductive anisotropic material layer along a major axis of symmetry is about 10 cm to about 45 cm.

6. The electrode assembly of claim 1, wherein, when viewed in the direction perpendicular to the front face of the conductive anisotropic material layer, an area enclosed by a perimeter of the conductive anisotropic material layer is at least 10% larger than an area enclosed by a perimeter of the electrode layer.

7. The electrode assembly of claim 1, wherein, when viewed in the direction perpendicular to the front face of the conductive anisotropic material layer, the conductive anisotropic material layer comprises:
a first slit extending from a first edge of the conductive anisotropic material layer toward a major axis of symmetry of the conductive anisotropic material layer; and
a second slit extending from a second edge of the conductive anisotropic material layer toward the major axis of symmetry of the conductive anisotropic material layer,
wherein the first edge is opposite the second edge,
wherein the first slit is opposite the second slit, and
wherein the first slit has a length equal to or within 20% of a length of the second slit.

8. The electrode assembly of claim 7, wherein the first slit and the second slit are both closer to the first end than the second end of the conductive anisotropic material layer.

9. The electrode assembly of claim 1, wherein the conductive anisotropic material layer is sandwiched between a first conductive adhesive or hydrogel layer and a second conductive adhesive or hydrogel layer, and wherein the conductive anisotropic material layer is or comprises a sheet of graphite.

10. The electrode assembly of claim 1, wherein the electrode assembly is adapted to be located on a back of the subject between shoulder blades of the subject.

11. An electrode assembly for delivering an alternating electric field to a subject, the electrode assembly comprising:
an electrode layer having a front face and a back face, the front face of the electrode layer adapted to face toward the subject, the back face being opposite to the front face,
wherein, when viewed in a direction perpendicular to the front face of the electrode layer, the electrode layer has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

12. The electrode assembly of claim 11, wherein the electrode layer has a single electrode element having the asymmetric oval, ovaloid, ovoid, or ovate shape or stretched asymmetric oval, ovaloid, ovoid, or ovate shape of the electrode layer.

13. The electrode assembly of claim 11, wherein the electrode layer comprises a first electrode element, a second electrode element, and a third electrode element,
wherein, when viewed in a direction perpendicular to the front face of the conductive anisotropic material layer,
the first electrode element has a substantially rounded triangular shape,
the second electrode element has a substantially rounded isosceles trapezoidal shape, and
the third electrode element has a substantially rounded half-circular shape.

14. An electrode assembly for delivering an alternating electric field to a subject, the electrode assembly comprising:
an electrode layer; and
a conductive anisotropic material layer electrically coupled to the electrode layer, the conductive anisotropic material layer having a front face and a back face, the back face of the conductive anisotropic material layer facing toward the electrode layer, the front face of the conductive anisotropic material layer adapted to face toward the subject,
wherein, when viewed in a direction perpendicular to the front face of the conductive anisotropic material layer, the conductive anisotropic material layer comprises:
a first edge, a second edge, and a third edge in a triangular configuration;
a first rounded vertex between the first edge and second edge,
a second rounded vertex between the second edge and third edge; and
a third rounded vertex between the third edge and first edge.

15. The electrode assembly of claim 14, wherein the first edge, the second edge, and the third edge are of substantially equal length, thereby forming an equilateral rounded triangular configuration.

16. The electrode assembly of claim 14, wherein the conductive anisotropic material layer is sandwiched between a first conductive adhesive or hydrogel layer and a second conductive adhesive or hydrogel layer, and wherein the conductive anisotropic material layer is or comprises a sheet of graphite.

17. The electrode assembly of claim 14, wherein, when viewed in the direction perpendicular to the front face of the conductive anisotropic material layer, one or more electrode elements of the electrode layer have a circular shape, or an oval, ovaloid, ovoid, or ovate shape, or a stretched oval, ovaloid, ovoid, or ovate shape, or an asymmetric oval, ovaloid, ovoid, or ovate shape, or a stretched asymmetric oval, ovaloid, ovoid, or ovate shape.

18. The electrode assembly of claim 14, wherein, when viewed in the direction perpendicular to the front face of the conductive anisotropic material layer, the electrode layer comprises:
a first circular-shaped or oval-, ovaloid-, ovoid-, or ovate-shaped, or stretched oval-, ovaloid-, ovoid-, or ovate-shaped, or asymmetric oval-, ovaloid-, ovoid-, or ovate-shaped or stretched asymmetric oval-, ovaloid-, ovoid-, or ovate-shaped electrode element aligned with the first rounded vertex of the conductive anisotropic material layer;
a second circular-shaped or oval-, ovaloid-, ovoid-, or ovate-shaped, or stretched oval-, ovaloid-, ovoid-, or ovate-shaped, or asymmetric oval-, ovaloid-, ovoid-, or ovate-shaped or stretched asymmetric oval-, ovaloid-, ovoid-, or ovate-shaped electrode element aligned with the second rounded vertex of the conductive anisotropic material layer; and
a third circular-shaped or oval-, ovaloid-, ovoid-, or ovate-shaped, or stretched oval-, ovaloid-, ovoid-, or ovate-shaped, or asymmetric oval-, ovaloid-, ovoid-, or ovate-shaped or stretched asymmetric oval-, ovaloid-, ovoid-, or ovate-shaped electrode element aligned with the third rounded vertex of the conductive anisotropic material layer.

19. The electrode assembly of claim 14, wherein, when viewed in the direction perpendicular to the front face of the conductive anisotropic material layer, the conductive anisotropic material layer has mirror symmetry about a major axis and comprises:
a first slit extending from the first edge of the conductive anisotropic material layer toward the major axis of the conductive anisotropic material layer; and
a second slit extending from the second edge of the conductive anisotropic material layer toward the major axis of the conductive anisotropic material layer,
wherein the first slit has a length equal to or within 20% of a length of the second slit.

20. The electrode assembly of claim 14, wherein at least one edge of the first edge, the second edge, and the third edge has a concave portion.
